(19) United States
(12) Patent Application Publication
(10) Pub. No.: US 2012/0122188 A1
(43) Pub. Date: May 17, 2012

(54) LIVE ORGANISM PRODUCT

(76) Inventor: JAMES B. WATSON, Pierce, NE (US)

(21) Appl. No.: 13/352,826

(22) Filed: Jan. 18, 2012

Related U.S. Application Data
(63) Continuation-in-part of application No. 12/287,181, filed on Oct. 7, 2008.
Application No. 12/287,181 is a continuation-in-part of application No. 11/700,464, filed on Jan. 31, 2007.
Application No. 11/700,464 is a continuation of application No. 10/853,346, filed on May 25, 2004, now abandoned.

(51) Int. Cl. C12N 1/20

(52) U.S. Cl. 435/252.1

(57) ABSTRACT

A live organism product wherein the organisms are in a dormant state and are suspended in a liquid carrier which is sufficiently devoid of moisture so that the organisms will remain in a dormant state for several months. The carrier is comprised of oil. The carrier may also include an absorbent. The product is stored and shipped in a plastic bag and is sprayed onto its target host or the like. The moisture and pH then activates the organisms.

### CROSS-REFERENCE TO RELATED APPLICATION

This is a continuation-in-part application of Petitioner's earlier continuation-in-part application Ser. No. 12/287,181, filed Oct. 7, 2008, entitled “LIVE ORGANISM PRODUCT,” which was a continuation-in-part application of Petitioners earlier continuation-in-part application Ser. No. 11/700,464, filed Jan. 31, 2007, entitled “LIVE BACTERIA PRODUCT,” which was a continuation application of Petitioner's earlier application Ser. No. 10/853,346, filed May 25, 2004, entitled “LIVE BACTERIA PRODUCT.”

### BACKGROUND OF THE INVENTION

1. Field of the Invention

This invention relates to a dormant organism product and more particularly to a live dormant organism product wherein live organisms, that are in a dormant state, are suspended in a clear liquid fluid carrier or medium that can be easily pumped and will stay in suspension for the life of the product. More particularly, the carrier, while being in a liquid state, is sufficiently devoid of moisture to prevent the organisms from becoming activated until the product is applied to feeds, forages, or directly onto animals such as livestock or the like. Hereinafter, feeds, forages, animals, livestock or the like will be referred to as “the target host”.

2. Description of the Related Art

Live bacteria formulations are frequently applied to forage products, feed, etc., to enhance fermentation and/or digestion. The prior art products fall generally into two categories which are either a dry soluble powder form or a dry granular non-soluble form. The dry granular products are directly applied to its target host through a metering device such as a Gandy box, usually at a rate of 4 to 16 ounces per ton of forage treated. The dry soluble products must first be hydrated with water, and then applied to its target host within a 48-hour time period post-hydration with application made through a liquid pump system at the rate of 37 grams to 1 gallon per ton of forage.

The dry granular products of the prior art, even if packaged in plastic pails, will sometimes come into direct contact with ambient air during manufacturing and processing or upon simply opening the product lid with the ambient air containing moisture or humidity. The moisture or humidity will activate the dormant bacteria upon contact which in turn causes the bacteria to inadvertently live and die before it can be applied to the forage. The value of the product will then be a total loss. The limestone carriers of the prior art products will carry minute traces of moisture, and such moisture severely reduces the product's ability to sustain an adequate shelf life. If the prior art product is a soluble powder product, it will also need to be hydrated before application. The hydration immediately activates all of the dormant bacteria and the producer has to apply the entire mixture within a 48-hour time frame or before since the bacteria will naturally deplete its food source and die, creating a total loss of the product value.

Further, the prior art products on the market today must be either removed from their original shipping container or physically poured into a dry applicator prior to application, or they must be contaminated with water, reducing their shelf life to less than 48 hours, post-contamination. Additionally, the prior art dry granular products have a tendency of sorting during shipment. This shakes any fines to the bottom of the shipping container leaving the either larger or lighter material to work its way to the top. Since the bacteria attach themselves to these particles, they may also migrate with these particles. Other dry soluble powder products may settle to the bottom of the applicator tank upon hydration. This will depend greatly on the product's rate of dilution. As application rates decrease to meet the producer's ongoing needs of hauling less product to the field, the manufacturers must in turn make their products more condensed to meet these needs. Such limits the solubility of the product.

The dry granular products of the prior art require a significant percentage of the product to be applied to the material which results in uniform coverage becoming difficult. Dry soluble powders mixed with water carriers have attempted to achieve low inclusion rates for some time. However, such products result in droplets which are very large in their molecular size and weight resulting in that there are actually few droplets being delivered per treatment, an example being one ton of forage treated with only ten large droplets so uniform coverage is poor.

One prior art reference is U.S. Pat. No. 4,161,397 issued Jul. 17, 1979 to Bellet et al. In the Bellet et al. composition, it appears that 99% of the bacteria die within one day and the other 1% of the bacteria die within 20 days. Further, in Bellet et al., the composition is applied to seeds which have fungicide thereon thereby making the treated seeds inappropriate for feeding to livestock or the like.

Another prior art reference is U.S. Pat. No. 4,518,696 (Gehrman et al). In the '696 Patent, the liquid carrier disclosed therein is devoid of polymers such as added to Applicant's liquid carrier to maintain the bacteria in suspension in the liquid carrier. As will be pointed out hereinafter, Applicant's product is sufficiently devoid of moisture so that substantially all the bacteria remain alive but in a dormant state for several months.

### SUMMARY OF THE INVENTION

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

A live bacteria product is disclosed wherein the live bacteria are in a dormant state and are suspended in a clear liquid fluid carrier or medium that may easily be pumped and will stay in suspension for the life of the product. More particularly, the carrier or medium for the live bacteria is comprised of a substantially moisture-free liquid which is comprised of: (1) mineral oil and polymers other than the bacteria; or (2) a processed oil obtained from either animal, vegetable or petroleum origins. In some cases, the carrier will also have a hydrophilic molecular sieve adsorbent (moisture scavenger) included therein.

The product of this invention is preferably applied to the target host at a micro-treatment rate of 2 to 28 grams per ton of target host being treated. In some cases, more or less percentages may be utilized. The live bacteria, by being suspended in the substantially moisture-free carrier, will remain dormant for several months, at least 7 to 12 months, until coming into contact with the target host at which time the moisture in the target host will cause the bacteria to be activated. The product of this invention is packaged in controlled shipping vessels such as polyurethane bags that reduce the risk of moisture contamination of any kind. The product is sold and shipped directly to the producer who will then take the collapsible bag of product and hook it directly to an applicator which applies the product to the target host. The target host may be forage, feed or the like. In some cases, the product may be sprayed directly onto animals such as chicks that will ingest the product while grooming themselves. Although the product is ideally an improved live bacterial product, the product may be freeze-dried live dormant organisms that require ambient moisture to begin proliferation including, but not limited to, modified live bacterial and viral vaccines such as Parvo, Bovine Rhinotracheitis, Leptospira, BVD, IBR and P13 or modified and non-modified pathogens.

The method of preparing the product is also described.

It is therefore a principal object of the invention to provide an improved live dormant organism product which requires ambient moisture to begin proliferation.

Yet another object of the invention is to provide a live dormant organism product wherein live organisms, that are in a dormant state, are suspended in a clear liquid fluid carrier or medium that can be easily pumped and will stay in suspension for the life of the product.

Still another object of the invention is to provide a live dormant organism product which is free of moisture to prevent the organism from becoming activated until the product is applied to the target host.

Still another object of the invention is to provide a live dormant organism product which may be applied to a target host at an extremely small rate.

Still another object of the invention is to provide a live dormant organism product wherein the carrier or medium therefore is comprised of a substantially moisture-free liquid comprised of: (1) mineral oil and polymers other than the bacteria; or (2) a processed oil obtained from either animal, vegetable or petroleum origins.

Still another object of the invention is to provide a live dormant organism product which will not be activated until coming into contact with moisture associated with the target host.

Yet another object of the invention is to provide a live dormant organism product which may be applied to forage or feed at a preferred micro-treatment rate.

A further object of the invention is to provide a live dormant organism product which is packaged in such a way to limit its exposure to possible contamination from outside sources, such as moisture-laden air or fluid water of any kind.

Still another object of the invention is to provide a live dormant organism product such as modified or non-modified live bacterial and viral vaccines.

Still another object of the invention is to provide a live dormant organism product which will remain alive but dormant for several months, i.e., seven to twelve months.

These and other objects will be apparent to those skilled in the art.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is a graph referred to as Graph A;

FIG. 2 is a graph referred to as Graph B;

FIG. 3 is a graph referred to as Graph C;

### DETAILED DESCRIPTION OF THE INVENTION

Embodiments are described more fully below with reference to the accompanying figures, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense in that the scope of the present invention is defined only by the appended claims.

In the following, the product of this invention is described as a live dormant bacteria product. However, it should be understood that the product could be: (1) a live dormant organism requiring ambient moisture to begin proliferation; (2) modified or live bacterial and viral vaccines such as Parvo, Bovine Rhinotracheitis, Leptospira, BVD, IBR and P13; or (3) modified and non-modified pathogens.

In this invention, conventional live viable harmless bacteria are mixed with a substantially moisture-free liquid carrier or medium. In general, the live bacteria used in the product of this invention will be any lactic acid producing bacteria that is permissible for use in animal related products. The United States Food and Drug Administration (FDA) and The American Association of Feed Control Officials (AAFCO) have published a list of the microorganism species which are “generally recognized as safe” (GRAS) for use in direct-fed microbial products. Table I hereinbelow lists presently approved bacteria for use with animal related products.

TABLE I
36.14 -- DFM Microorganisms under the “GRAS” Status

| | |
|---|---|
| Aspergillus niger | Lactobacillus curvatus |
| Aspergillus oryzae | Lactobacillus delbruekii |
| Bacillus coagulans | Lactobacillus fermentum |
| Bacillus lentus | Lactobacillus helveticus |
| Bacillus licheniformis | Lactobacillus lactis |
| Bacillus pumilus | Lactobacillus plantarum |
| Bacillus subtilis | Lactobacillus euterii |
| Bacteroides amylophilus | Leuconostoc mesenteroides |
| Bacteroides capillosus | Pediococcus acidilacticii |
| Bacteroides ruminocola | Pediococcus cervisiae |
| Bacteroides suis | Pediococcus pentosaceus |
| Bifidobacterium adolescentis | Propionibacterium freudenreichii |
| Bifidobacterium animalis | Propionibacterium shermanii |
| Bifidobacterium bifidum | Saccharomyces cerevisiae |
| Bifidobacterium infantis | Entercococcus cremoris |
| Bifidobacterium longum | Entercococcus diacetylactis |
| Bifidobacterium thermophilum | Entercococcus faecium |
| Lactobacillus acidolphilus | Entercococcus intermedius |
| Lactobacillus brevis | Entercococcus lactis |
| Lactobacillus buchneri | Entercococcus thermophilius |
| Lactobacillus bulgaricus | Lactobacillus cellobiosus |
| Lactobacillus casei | |

Preferably, the liquid carrier is comprised of a gelled mineral oil which has been dewatered or dehydrated to remove the moisture therefrom. One such liquid carrier is a product marketed under the trademark SYNERGEL™, formerly manufactured by Penreco of 138 Petroila Street, Karnes City, Pa. 16041. Penreco has been acquired by Calumet Specialty Products. The SYNERGEL product is marketed by Calumet Penreco. The SYNERGEL product of Calumet Penreco is also marketed by Holland Applied Technologies. Other oils such as vegetable oil may be used as long as they are substantially moisture free since the end product preferably contains one percent or less moisture.

As will be explained in more detail, a suspension agent such as a polymer or polymers are added to the oil to thicken the oil to increase the suspension characteristics of the product to ensure that the bacteria will remain in suspension in the oil. A suitable polymer is “Benzene, ethyl-, polymer with 2-methyl-1,3-butadiene, hydrogenated.” A moisture scavenger or absorbent may also be added to the oil-polymer mixture to absorb moisture in the product. A suitable absorbent will be described hereinafter.

The method of preparing the liquid carrier will now be described. The mineral or vegetable oil (hereinafter “oil”) is mixed with the polymer. If a moisture scavenger is being used, it will also be added to the oil and polymer mixture. The mixture is slowly stirred and heated to at least 85 degrees centigrade. When the mixture reaches 85 degrees C., the mixture is subjected to a negative pressure and rapidly mixed to homogenize or shear the components of the mixture together. The mixture is then cooled to 70 degrees F. before adding the typically 100 mesh bacteria. The bacteria is then blended into the cooled mixture by again pulling a negative pressure while stirring and shearing the bacteria into the mixture. It is important to pull a negative pressure or vacuum prior to the blending procedure to keep air bubbles out of the mixture since the air bubbles may contain water vapor.

Tables A and B illustrate the preferred ranges of the ingredients of the product of this invention.

TABLE A

| Ingredient | Range |
|---|---|
| Vegetable Oil | 91-94% by volume |
| Polymer | 4-6% by volume |
| Moisture Scavenger | 0-3% by volume |

TABLE B

| Ingredient | Range |
|---|---|
| Mineral Oil | 91-94% by volume |
| Polymer | 4-6% by volume |
| Moisture Scavenger | 0-3% by volume |

Other suspension agents may also be used from time to time to reduce the possibility of the settling of the bacteria therein. The product of this invention is designed to only activate the bacteria at the physical point of contact with the target host. Upon contact, the target host ambient moisture content will activate (i.e., initiate the life cycle thereof) the dormant bacteria found within the liquid microbial medium or carrier.

Upon receipt of the packaged carrier, applicant may then add a moisture scavenger product such as a hydrophilic molecular sieve adsorbent as an insurance program. The adsorbent may comprise a natural or synthetic zeolite consisting of crystalline metal aluminosilicate, alkali metal aluminosilicate or sodium aluminosilicate. Finally, whatever bacteria are needed for a particular purpose will then be added. The final blend is then packaged in moisture and ultraviolet retardant containers such as collapsible polyurethane bags, very similar to I.V. bags. The product is then sold and shipped directly to the retailer and/or end user. The end user will then take the collapsible bag of liquid microbial blend and hook it directly to an applicator so that extremely small droplets are created which are sprayed upon the target host.

The invention described herein provides the ability to inoculate other products with dormant live bacteria, by means of a light spraying application. Whereas the bacteria is protected with the oil/polymer blend allowing the host bacteria to survive longer in a non-favorable environment, the coating of the bacteria cell walls with the oil/polymer covering provides a physical chemical moisture barrier. The above provides the ability to permit the live bacteria to be introduced onto a pelleted feed without immediately inadvertently activating the oil/polymer/host bacteria through ambient moisture contamination from the feed itself.

The product of this invention permits it to be applied to its target host at a preferred micro-treatment rate of 2 to 28 grams per one ton of forage or the material treated (with no additional water or carriers needed). In some cases, higher or lower ratios may be employed. Such micro-treatment is substantially less than prior art products that have treatment rates that range from 37.88 grams to 8.3453 pounds per one ton of forage treated.

The product of this invention and its packaging limits its exposure to possible contamination from outside sources, such as moisture-laden air or fluid water of any kind. The product of this invention is designed to only activate at the physical point of contact with the target host. Upon contact with the target host, the change in pH and moisture activates the dormant bacteria.

To remain 100% alive, freeze-dried bacteria must be kept free of water activity, kept out of direct ultra'violet light, and maintained in temperatures less than 110° F. The best place to meet all of these criteria is to keep the bacteria in liquid nitrogen or a freezer. If kept frozen, the bacteria will remain alive and viable for many years. Unfortunately, if you want to apply these bacteria to agricultural needs, the bacteria must be taken out of the freezer and put into other types of carriers. In doing so, the bacteria are inadvertently exposed to ambient water activity.

Manufacturers have used oils for their residual effect and as a carrier for the freeze-dried bacteria under the assumption that oil would protect the bacteria from ambient moistures, thereby extending the life span of the bacteria. However, when the bacteria are placed in a feed grade mineral oil, the bacteria quickly die off in about 30 days. Another noted problem is that the bacteria in oil-based carriers quickly settle out, since the bacteria are non-soluble in oil.

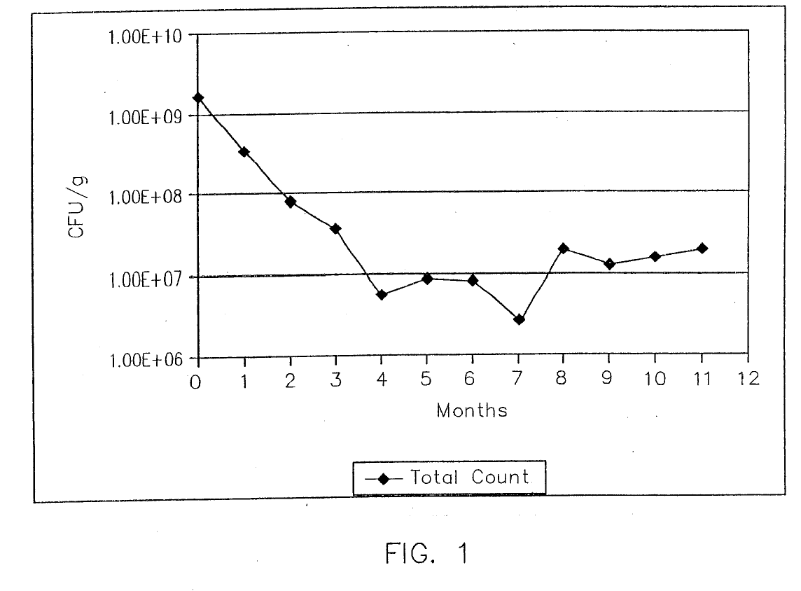

In developing the product described in this application, Applicant realized the problems with putting freeze-dried bacteria in an oil-based carrier. Testing showed that the bacteria in a feed grade mineral oil had a very short life span as seen in FIG. 1. In FIG. 1, a one-log die off represents a 90% die off of live bacteria. For example, (1.00 E 09-to-1.00 E 08)=a 90% loss of live bacteria. A two-log loss, for example, (1.00 E 09-to-1.00 E07)=a 99% loss of live bacteria. A three-log loss represents a 99.9% loss of live bacteria and so on.

FIG. 1 demonstrates the life span of freeze-dried Lactobacillius acidolphilus bacteria blended into a feed grade mineral oil. Please note that there was a 78.92% die off of the La bacteria in the first month, a 95.07% die off on the second month and a 97.78% total cumulative die off of the La bacteria upon the third month with a 99.67% die off on the fourth month.

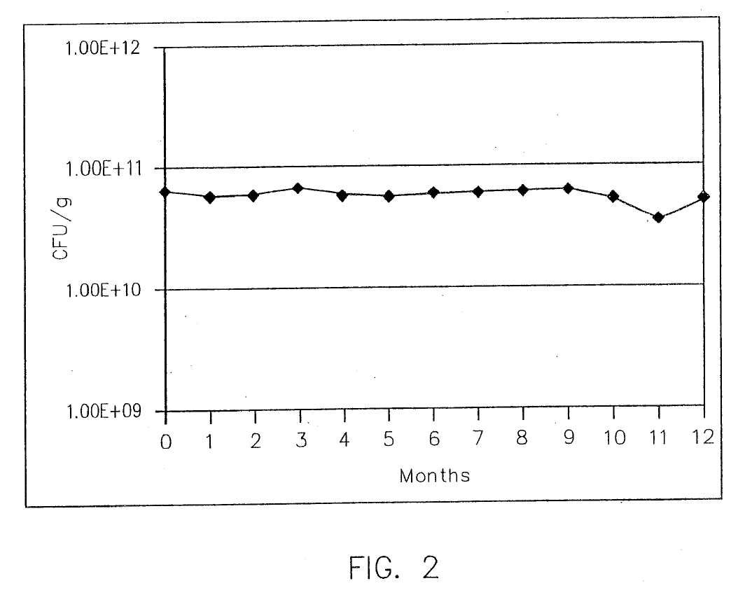

FIG. 2 illustrates a test of Lactobacillius acidolphilus bacteria blended into the carrier product described and claimed in this application and it can be seen that the bacteria in FIG. 2 lived for at least 12 months time, at which time the test was discontinued.

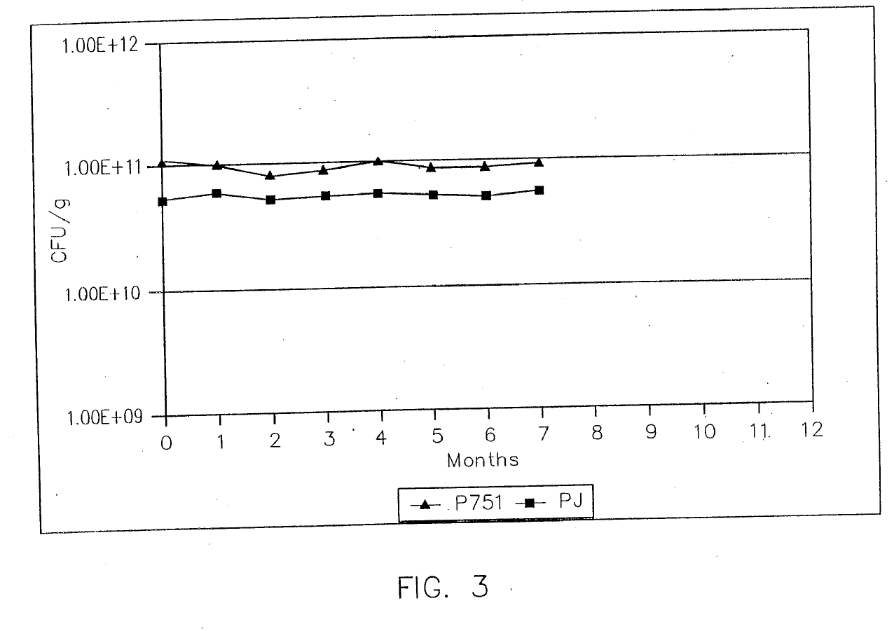

FIG. 3 is a currently ongoing experiment, scheduled for 12 months testing. In its seventh month of testing, FIG. 3 shows two bacteria being separately tested in the product described and claimed in this application. In the tests of FIG. 3, (P751) Pediococcus acidilaticii and (PJ) Pediococcus pentosaceus were still 100% alive at 7 months time.

It can be seen from FIGS. 2 and 3, wherein the carrier of this application was utilized, the life span of the freeze-dried bacteria is greatly extended than that previously possible.

In use, the live bacteria product of admixture is applied to livestock feed with the admixture continuing to prevent the dormant bacteria from hydration for a period of time up to at least 40 days. The treated livestock feed is fed to livestock so that the treated livestock feed is subjected to a low pH intestinal environment, which breaks down the protective coating on the bacteria to cause the bacteria to become hydrated by ambient moisture in the digestive system of the livestock to begin the bacteria's life cycle.

Thus it can be seen that the invention accomplishes at least all of its stated objectives.

Although the invention has been described in language that is specific to certain structures and methodological steps, it is to be understood that the invention defined in the appended claims is not necessarily limited to the specific structures and/or steps described. Rather, the specific aspects and steps are described as forms of implementing the claimed invention. Since many embodiments of the invention can be practiced without departing from the spirit and scope of the invention, the invention resides in the claims hereinafter appended.

1. The method of forming stabilized admixtures comprising the steps of:
proving a liquid carrier which consists of 91-94% by volume dewatered oil, 4-6% by volume polymers, and 0-3% by volume moisture scavenger;
mixing live, dormant bacteria with the liquid carrier.

2. The method of claim 1 wherein the moisture scavenger comprises an adsorbent.

3. The method of claim 1 wherein the moisture scavenger comprises zeolite.

4. The method of claim 1 wherein the moisture scavenger is comprised of crystalline metal aluminosilicate, alkali metal aluminosilicate or sodium aluminosilicate.

5. The method of claim 1 wherein the moisture scavenger comprises a hydrophilic molecular sieve adsorbent.

6. The method of claim 1 wherein substantially all the bacteria will remain in a dormant state for at least twelve months.

7. The method of claim 1 wherein the bacteria is a lactic acid producing bacteria.

8. The method of claim 1 wherein the oil is heated prior to the suspension agent being added thereto.

9. The method of claim 1 wherein the oil is a vegetable oil.

10. The method of claim 1 wherein the polymer comprises Benzene, ethyl-, polymer with 2-methyl-1,3-butadiene, hydrogenated.

11. The method of claim 1 wherein the admixture comprises 91-94% by volume vegetable oil, 4-6% by volume polymer and 0-3% by volume moisture scavenger.

12. The method of claim 1 wherein the admixture comprises 91-94% by volume mineral oil, 4-6% by volume polymer and 0-3% by volume moisture scavenger.
